(19) United States
(12) Patent Application Publication
(10) Pub. No.: US 2009/0202562 A1
(43) Pub. Date: Aug. 13, 2009

(54) ANTI-GFRALPHA3 ANTIBODIES

(75) Inventors: Dane S. Worley, Southborough, MA (US); Dinah Wen-Yee Sah, Boston, MA (US); Katherine W. Seamans, Dallas, TX (US)

Correspondence Address: FISH & RICHARDSON P.C., P.O. BOX 1022, MINNEAPOLIS, MN, 55440-1022, US

(73) Assignee: BIOGEN IDEC MA INC., Cambridge, MA (US)

(21) Appl. No.: 11/917,676

(22) PCT Filed: Jun. 16, 2006
(86) PCT No.: PCT/US06/23821

§ 371 Date: Sep. 23, 2008

Related U.S. Application Data
(60) Provisional application No. 60/691,850, filed on Jun. 17, 2005.

(51) Int. Cl. A61K 39/395; C12N 5/00; C12N 5/02; C07K 16/28

(52) U.S. Cl. 424/158.1; 435/346; 435/375; 530/387.1; 530/387.3; 530/388.2; 530/389.2

(57) ABSTRACT

Antibodies and antibody fragments that bind to the receptor GFRalpha3 and inhibit formation of a Neublastin-GFRalpha3-Ret ternary complex are disclosed. Also disclosed are methods of using the antibodies and antibody fragments to inhibit phosphorylation of Ret in a cell and treat disorders and in a subject.

### TECHNICAL FIELD

The invention relates to antibodies and antibody fragments that bind to the receptor GFRalpha3.

### BACKGROUND

Ret is a transmembrane receptor tyrosine kinase expressed in neuroendocrine cells and in certain neuroendocrine tumors. Activating Ret mutations occur in the inherited cancer syndrome multiple endocrine neoplasia type 2 and in a subset of the related sporadic tumors, medullary thyroid carcinoma and pheochromocytoma (both derived from neuroendocrine tissues).

Ret is a receptor for the neurotrophic factors Glial-Derived Neurotrophic Factor (GDNF), Neurturin, Neublastin (also known as Artemin and Enovin), and Persephin. Ligand specificity is conferred by binding of a neurotrophic factor to a particular GDNF family receptor alpha (GFRalpha). The GFRalpha1 to GFRalpha4 co-receptors are glycosyl-phosphatidyl inositol (GPI) anchored proteins that, when bound to a preferred neurotrophic factor, activate Ret. GDNF binds preferentially to GFRalpha1, Neurturin binds preferentially to GFRalpha2, Neublastin binds preferentially to GFRalpha3 (also known as RetL3), and Persephin binds preferentially to GFRalpha4.

Once activated, Ret recruits a variety of signaling molecules that mediate biological responses. Ret can activate various signaling pathways, such as RAS/extracellular signal-regulated kinase (ERK), phosphatidylinositol 3-kinase (PI3K)/AKT, p38 mitogen-activated protein kinase (MAPK), and c-Jun N-terminal kinase (JNK) pathways. These signaling pathways are activated via binding of adaptor proteins to intracellular tyrosine residues of Ret phosphorylated by its own kinase activity.

Neublastin binding to GFRalpha3 and Ret forms a ternary signaling complex (Baudet et al. 2000, Development, 127:4335; Baloh et al., 1998, Neuron, 21:1291) localized predominantly on nociceptive sensory neurons (Orozco et al., 2001, Eur. J. Neurosci., 13(11):2177). Neublastin promotes the survival of neurons of the peripheral and central nervous system such as dopaminergic neurons (Baudet et al., 2000, Development, 127:4335; Rosenblad et al., 2000, Mol. Cell Neurosci., 15(2):199). Thus, Neublastin, GFRalpha3, and Ret are relevant to the treatment of neuropathy and more specifically in the treatment of neuropathic pain.

### SUMMARY

The invention is based, at least in part, on the identification and characterization of an antibody fragment that binds to GFRalpha3 and inhibits formation of a Neublastin-GFRalpha3-Ret ternary complex.

In one aspect, the invention features an isolated antibody or antigen-binding fragment thereof that selectively binds to GFRalpha3 and inhibits formation of a Neublastin-GFRalpha3-Ret ternary complex.

The term “isolated” refers to a molecule that is substantially free of its natural environment. For instance, an isolated antibody is substantially free of cellular material from the cell or tissue source from which it was derived. The term also refers to preparations where the isolated antibody is sufficiently pure for a pharmaceutical composition, or at least 70-80% (w/w) pure, at least 80-90% (w/w) pure, at least 90-95% (w/w) pure, or at least 95%, 96%, 97%, 98%, 99%, or 100% (w/w) pure.

The term “antibody or antigen-binding fragment thereof” encompasses proteins that include at least one immunoglobulin variable region, e.g., an amino acid sequence that provides an immunoglobulin variable domain or immunoglobulin variable domain sequence. For example, the term includes an antigen-binding protein that has a heavy (H) chain variable region (abbreviated herein as VH), and a light (L) chain variable region (abbreviated herein as VL). In another example, the term includes an antigen binding protein that includes two heavy (H) chain variable regions and two light (L) chain variable regions. The term encompasses antigen-binding fragments of antibodies (e.g., single chain antibodies, Fab fragments, F(ab′)2 fragments, Fd fragments, Fv fragments, and dAb fragments) as well as complete antibodies, e.g., intact immunoglobulins of types IgA, IgG, IgE, IgD, IgM (as well as subtypes thereof). The light chains of the immunoglobulin may be of types kappa or lambda. In some embodiments, the antibody is glycosylated. An antibody can be functional for antibody-dependent cytotoxicity and/or complement-mediated cytotoxicity, or may be non-functional for one or both of these activities. The VH and VL regions can be further subdivided into regions of hypervariability, termed “complementarity determining regions” (“CDR”), interspersed with regions that are more conserved, termed “framework regions” (FR). The extent of the FR's and CDR's has been precisely defined (see, Kabat, E. A., et al. (1991) Sequences of Proteins of Immunological Interest, Fifth Edition, US Department of Health and Human Services, NIH Publication No. 91-3242; and Chothia, C. et al. (1987) J. Mol. Biol. 196:901-917). Kabat definitions are used herein. Each VH and VL is typically composed of three CDR's and four FR's, arranged from amino-terminus to carboxyl-terminus in the following order: FR1, CDR1, FR2, CDR2, FR3, CDR3, FR4.

The term “selectively binds” refer to two molecules forming a complex that is relatively stable under physiologic conditions. Selective binding is characterized by a high affinity and a low to moderate capacity as distinguished from nonspecific binding which usually has a low affinity with a moderate to high capacity. Typically, binding is considered selective when the antibody binds with a Kd of less than 10-6 M. If necessary, nonspecific binding can be reduced without substantially affecting selective binding by varying the binding conditions.

Also disclosed is an isolated antibody or antigen-binding fragment thereof that selectively binds to GFRalpha3 and crossblocks binding of the antibody MOR02683.

Also disclosed is an isolated antibody or antigen-binding fragment thereof that selectively binds to GFRalpha3 on the same epitope as the antibody MOR02683.

Also disclosed is an isolated antibody or antigen-binding fragment thereof that selectively binds to GFRalpha3 and comprises a VH domain that is at least 80% identical to the amino acid sequence of SEQ ID NO:1. In some embodiments, the VH domain is at least 90% identical to the amino acid sequence of SEQ ID NO:1. In some embodiments, the VH domain is at least 95% identical to the amino acid sequence of SEQ ID NO:1. In some embodiments, the VH domain is identical to the amino acid sequence of SEQ ID NO:1.

Also disclosed is an isolated antibody or antigen-binding fragment thereof that selectively binds to GFRalpha3 and comprises a VL domain that is at least 80% identical to the amino acid sequence of SEQ ID NO:2. In some embodiments, the VL domain is at least 90% identical to the amino acid sequence of SEQ ID NO:2. In some embodiments, the VL domain is at least 95% identical to the amino acid sequence of SEQ ID NO:2. In some embodiments, the VL domain is identical to the amino acid sequence of SEQ ID NO:2.

Also disclosed is an isolated antibody or antigen-binding fragment thereof that selectively binds to GFRalpha3 and comprises (i) a VH domain that is at least 80% identical to the amino acid sequence of SEQ ID NO:1, and (ii) a VL domain that is at least 80% identical to the amino acid sequence of SEQ ID NO:2. In some embodiments, (i) the VH domain is at least 90% identical to the amino acid sequence of SEQ ID NO:1, and (ii) the VL domain is at least 90% identical to the amino acid sequence of SEQ ID NO:2. In some embodiments, (i) the VH domain is at least 95% identical to the amino acid sequence of SEQ ID NO:1, and (ii) the VL domain is at least 95% identical to the amino acid sequence of SEQ ID NO:2. In some embodiments, (i) the VH domain is identical to the amino acid sequence of SEQ ID NO:1, and (ii) the VL domain is identical to the amino acid sequence of SEQ ID NO:2.

Also disclosed is an isolated antibody or antigen-binding fragment thereof that selectively binds to GFRalpha3 and comprises a VH domain comprising a heavy chain complementarity determining region (CDR) that is at least 90% identical to the amino acid sequence of SEQ ID NO:3, SEQ ID NO:4, or SEQ ID NO:5. In some embodiments, the VH domain comprises a first heavy chain CDR that is at least 90% identical to the amino acid sequence of SEQ ID NO:3, a second heavy chain CDR that is at least 90% identical to the amino acid sequence of SEQ ID NO:4, and a third heavy chain CDR that is at least 90% identical to the amino acid sequence of SEQ ID NO:5. In some embodiments, the VH domain comprises the amino acid sequences of SEQ ID NO:3, SEQ ID NO:4, and SEQ ID NO:5.

Also disclosed is an isolated antibody or antigen-binding fragment thereof that selectively binds to GFRalpha3 and comprises a VL domain comprising a light chain CDR that is at least 90% identical to the amino acid sequence of SEQ ID NO:6, SEQ ID NO:7, or SEQ ID NO:8. In some embodiments, the VL domain comprises a first light chain CDR that is at least 90% identical to the amino acid sequence of SEQ ID NO:6, a second light chain CDR that is at least 90% identical to the amino acid sequence of SEQ ID NO:7, and a third light chain CDR that is at least 90% identical to the amino acid sequence of SEQ ID NO:8. In some embodiments, the VL domain comprises the amino acid sequences of SEQ ID NO:6, SEQ ID NO:7, and SEQ ID NO:8.

Also disclosed is an isolated antibody or antigen-binding fragment thereof that selectively binds to GFRalpha3 and comprises (i) a VH domain comprising a heavy chain CDR that is at least 90% identical to the amino acid sequence of SEQ ID NO:3, SEQ ID NO:4, or SEQ ID NO:5, and (ii) a VL domain comprising a light chain CDR that is at least 90% identical to the amino acid sequence of SEQ ID NO:6, SEQ ID NO:7, or SEQ ID NO:8. In some embodiments, (i) the VH domain comprises a first heavy chain CDR that is at least 90% identical to the amino acid sequence of SEQ ID NO:3, a second heavy chain CDR that is at least 90% identical to the amino acid sequence of SEQ ID NO:4, and a third heavy chain CDR that is at least 90% identical to the amino acid sequence of SEQ ID NO:5, and (ii) the VL domain comprises a first light chain CDR that is at least 90% identical to the amino acid sequence of SEQ ID NO:6, a second light chain CDR that is at least 90% identical to the amino acid sequence of SEQ ID NO:7, and a third light chain CDR that is at least 90% identical to the amino acid sequence of SEQ ID NO:8. In some embodiments, (i) wherein the VH domain comprises the amino acid sequences of SEQ ID NO:3, SEQ ID NO:4, and SEQ ID NO:5, and (ii) the VL domain comprises the amino acid sequences of SEQ ID NO:6, SEQ ID NO:7, and SEQ ID NO:8.

In some embodiments, an isolated antibody or antigen-binding fragment thereof described herein is a humanized antibody.

In some embodiments, an isolated antibody or antigen-binding fragment thereof described herein is a fully human antibody.

In some embodiments, an isolated antibody or antigen-binding fragment thereof described herein is a monoclonal antibody.

In some embodiments, an isolated antibody or antigen-binding fragment thereof described herein is a single chain antibody.

In some embodiments, an isolated antibody or antigen-binding fragment thereof described herein is a polyclonal antibody, a chimeric antibody, an F_{ab} fragment, an F_{(ab′)2} fragment, an F_{ab′} fragment, an F_{sc} fragment, or an Fᵥ fragment.

Also disclosed is an isolated cell that produces an antibody or antigen-binding fragment thereof described herein. The cell can be, for example, a fused cell obtained by fusing a mammalian B cell and myeloma cell.

Also disclosed is a pharmaceutical composition comprising (i) an antibody or antigen-binding fragment thereof described herein, and (ii) a pharmaceutically acceptable carrier.

Also disclosed is a method of inhibiting formation of a Neublastin-GFRalpha3-Ret ternary complex in a cell, the method comprising contacting a cell expressing GFRalpha3 with an amount of an antibody or antigen-binding fragment thereof described herein effective to inhibit formation of a Neublastin-GFRalpha3-Ret ternary complex.

Also disclosed is a method of inhibiting Ret phosphorylation in a cell, the method comprising contacting a cell expressing GFRalpha3 with an amount of an antibody or antigen-binding fragment thereof described herein effective to inhibit Ret phosphorylation.

Also disclosed is a method of treating cancer in a subject, the method comprising administering to a subject (e.g., a human) in need thereof a pharmaceutical composition comprising an effective amount of an antibody or antigen-binding fragment thereof described herein.

As used herein, the terms “to treat,” “treating,” and “treatment” refer to administering a therapy in an amount, manner, and/or mode effective to improve or ameliorate a symptom or parameter that characterizes a pathological condition, to reduce the severity of a symptom or parameter that characterizes a pathological condition, to prevent, slow or reverse progression of the pathological condition, or to prevent one or more symptom or parameter of the pathological condition.

Also disclosed is a method of determining Neublastin binding affinity, the method comprising: providing a cell expressing GFRalpha3 and Ret; contacting the cell with Neublastin; incubating the cell in the presence of Neublastin; contacting the cell with an antibody or antigen-binding fragment thereof described herein; incubating the cell in the presence of the antibody or antigen-binding fragment thereof; measuring the amount of the antibody or antigen-binding fragment thereof bound to the cell; and determining the binding affinity of Neublastin to GFRalpha3 and Ret on the cell as a factor of the measured amount of binding of the antibody or antigen-binding fragment thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is an alignment of the amino acid sequences of human, rat, and murine GFRalpha3.

FIGS. 2A and 2B are plots depicting detection of expression of rat GFRalpha3 by the R11 polyclonal antibody (FIG. 1A) or the Fab fragment MOR02683 (FIG. 1B).

FIG. 3 is a plot depicting detection of expression of human and murine GFRalpha3 by the Fab fragment MOR02683.

FIG. 4 is a graph depicting concentration-dependent inhibition of Neublastin-GFRalpha3-Ret ternary complex formation by the Fab fragment MOR02683.

FIG. 5 is a graph depicting induction of Ret phosphorylation by increasing concentrations of Neublastin.

FIG. 6 is a graph depicting concentration-dependent inhibition of Neublastin-induced Ret phosphorylation by the Fab fragment MOR02683.

FIG. 7 is a graph depicting concentration-dependent inhibition of Neublastin-induced ERK phosphorylation by the Fab fragment MOR02683.

FIG. 8 is a graph depicting a dose-response curve in a Neublastin competition binding assay applying the blocking anti-GFRalpha3 Fab MOR02683 (▪) or the non-blocking anti-GFRalpha3 Fab MOR02682 ().

### DETAILED DESCRIPTION

The present invention provides antibodies and antigen-binding fragments thereof that bind to GFRalpha3 and inhibit formation of a Neublastin-GFRalpha3-Ret ternary complex.

### Antibody Generation

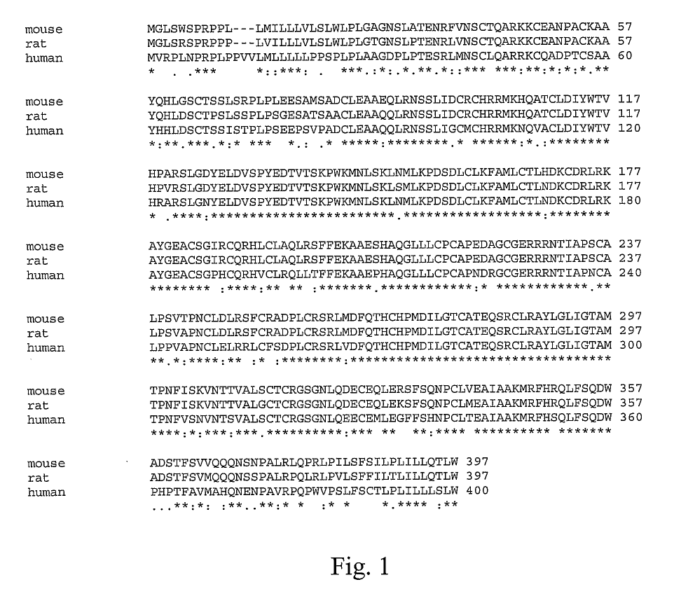

Antibodies or antibody fragments that bind to GFRalpha3 can be generated by immunization, e.g., using an animal, or by in vitro methods such as phage display. A polypeptide that includes all or part of GFRalpha3 can be used to generate an antibody or antibody fragment. An alignment of the amino acid sequences of human (SEQ ID NO:9; GenBank™ Accession 060609), rat (SEQ ID NO:10; GenBank™ Accession NP_{—}445850), and murine (SEQ ID NO:11; GenBank™ Accession 035118) GFRalpha3 is depicted in FIG. 1 (* indicates those amino acid residues conserved among all three species). Amino acids 1-31 of SEQ ID NO:9 correspond to a predicted signal sequence of human GFRalpha3. In some embodiments, a portion of the mature GFRalpha3 polypeptide (e.g., the extracellular region lacking the GPI linkage sequence) can be used as an immunogen to generate antibodies that can be screened for reactivity to GFRalpha3. In some embodiments, a cell expressing all or part of GFRalpha3 can be used as an immunogen to generate antibodies.

In some embodiments, an immunized animal contains immunoglobulin producing cells with natural, human, or partially human immunoglobulin loci. In some embodiments, the non-human animal includes at least a part of a human immunoglobulin gene. For example, it is possible to engineer mouse strains that are deficient in mouse antibody production and contain large fragments of the human Ig loci. Using hybridoma technology, antigen-specific monoclonal antibodies derived from the genes with the desired specificity can be produced and selected. See, e.g., XenoMouse™, Green et al. Nature Genetics 7:13-21 (1994), US 2003-0070185, U.S. Pat. No. 5,789,650, and WO 96/34096.

Non-human antibodies to GFRalpha3 can also be produced, e.g., in a rodent. The non-human antibody can be humanized, e.g., as described in U.S. Pat. No. 6,602,503, EP 239 400, U.S. Pat. No. 5,693,761, and U.S. Pat. No. 6,407,213.

EP 239 400 (Winter et al.) describes altering antibodies by substitution (within a given variable region) of their CDRs for one species with those from another. CDR-substituted antibodies can be less likely to elicit an immune response in humans compared to true chimeric antibodies because the CDR-substituted antibodies contain considerably less non-human components. See Riechmann et al., 1988, Nature 332, 323-327; Verhoeyen et al., 1988, Science 239, 1534-1536. Typically, CDRs of a murine antibody are substituted into the corresponding regions in a human antibody by using recombinant nucleic acid technology to produce sequences encoding the desired substituted antibody. Human constant region gene segments of the desired isotype (e.g., gamma I for CH and kappa for CL) can be added and the humanized heavy and light chain genes can be co-expressed in mammalian cells to produce soluble humanized antibody.

WO 90/07861 describes a process that includes choosing human V framework regions by computer analysis for optimal protein sequence homology to the V region framework of the original murine antibody, and modeling the tertiary structure of the murine V region to visualize framework amino acid residues that are likely to interact with the murine CDRs. These murine amino acid residues are then superimposed on the homologous human framework. See also U.S. Pat. Nos. 5,693,762; 5,693,761; 5,585,089; and 5,530,101. Tempest et al., 1991, Biotechnology 9, 266-271 use, as standard, the V region frameworks derived from NEWM and REI heavy and light chains, respectively, for CDR-grafting without radical introduction of mouse residues. An advantage of using the Tempest et al. approach to construct NEWM and REI based humanized antibodies is that the three dimensional structures of NEWM and REI variable regions are known from x-ray crystallography and thus specific interactions between CDRs and V region framework residues can be modeled.

Non-human antibodies can be modified to include substitutions that insert human immunoglobulin sequences, e.g., consensus human amino acid residues at particular positions, e.g., at one or more (preferably at least five, ten, twelve, or all) of the following positions: (in the framework of the variable domain of the light chain) 4L, 35L, 36L, 38L, 43L, 44L, 58L, 46L, 62L, 63L, 64L, 65L, 66L, 67L, 68L, 69L, 70L, 71L, 73L, 85L, 87L, 98L, and/or (in the framework of the variable domain of the heavy chain) 2H, 4H, 24H, 36H, 37H, 39H, 43H, 45H, 49H, 58H, 60H, 67H, 68H, 69H, 70H, 73H, 74H, 75H, 78H, 91H, 92H, 93H, and/or 103H (according to the Kabat numbering). See, e.g., U.S. Pat. No. 6,407,213.

Fully human monoclonal antibodies that bind to GFRalpha3 can be produced, e.g., using in vitro-primed human splenocytes, as described by Boerner et al., 1991, J. Immunol., 147, 86-95. They may be prepared by repertoire cloning as described by Persson et al., 1991, Proc. Nat. Acad. Sci. USA, 88: 2432-2436 or by Huang and Stollar, 1991, J. Immunol. Methods 141, 227-236; also U.S. Pat. No. 5,798,230. Large nonimmunized human phage display libraries may also be used to isolate high affinity antibodies that can be developed as human therapeutics using standard phage technology (see, e.g., Vaughan et al, 1996; Hoogenboom et al. (1998) Immunotechnology 4:1-20; and Hoogenboom et al. (2000) Immunol Today 2:371-8; US 2003-0232333).

As used herein, an “immunoglobulin variable domain sequence” refers to an amino acid sequence that can form the structure of an immunoglobulin variable domain. For example, the sequence may include all or part of the amino acid sequence of a naturally-occurring variable domain. For example, the sequence may omit one, two or more N- or C-terminal amino acids, internal amino acids, may include one or more insertions or additional terminal amino acids, or may include other alterations. In one embodiment, a polypeptide that includes an immunoglobulin variable domain sequence can associate with another immunoglobulin variable domain sequence to form a target binding structure (or “antigen binding site”), e.g., a structure that interacts with GFRalpha3.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains. The heavy and light immunoglobulin chains can be connected by disulfide bonds. The heavy chain constant region typically includes three constant domains, CH1, CH2 and CH3. The light chain constant region typically includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

One or more regions of an antibody can be human, effectively human, or humanized. For example, one or more of the variable regions can be human or effectively human. For example, one or more of the CDRs, e.g., heavy chain (HC) CDR1, HC CDR2, HC CDR3, light chain (LC) CDR1, LC CDR2, and LC CDR3, can be human. Each of the light chain CDRs can be human. HC CDR3 can be human. One or more of the framework regions (FR) can be human, e.g., FR1, FR2, FR3, and FR4 of the HC or LC. In some embodiments, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. One or more of the constant regions can be human, effectively human, or humanized. In another embodiment, at least 70, 75, 80, 85, 90, 92, 95, or 98% of the framework regions (e.g., FR1, FR2, and FR3, collectively, or FR1, FR2, FR3, and FR4, collectively) or the entire antibody can be human, effectively human, or humanized. For example, FR1, FR2, and FR3 collectively can be at least 70, 75, 80, 85, 90, 92, 95, 98, or 99% identical to a human sequence encoded by a human germline segment.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified such that the modified form elicits less of an immune response in a human than does the non-modified form, e.g., is modified to include a sufficient number of human framework amino acid positions such that the immunoglobulin variable region does not elicit an immunogenic response in a normal human. Descriptions of “humanized” immunoglobulins include, for example, U.S. Pat. No. 6,407,213 and U.S. Pat. No. 5,693,762. In some cases, humanized immunoglobulins can include a non-human amino acid at one or more framework amino acid positions.

All or part of an antibody can be encoded by an immunoglobulin gene or a segment thereof. Exemplary human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids).

The term “antigen-binding fragment” of a full length antibody refers to one or more fragments of a full-length antibody that retain the ability to specifically bind to a target of interest (i.e., GFRalpha3). Examples of binding fragments encompassed within the term “antigen-binding fragment” of a full length antibody include: (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody; (v) a dAb fragment (Ward et al., (1989) Nature 341:544-546), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., Bird et al. (1988) Science 242:423-426; and Huston et al. (1988) Proc. Natl. Acad. Sci. USA 85:5879-5883.

### Variants of MOR02683

As disclosed in the accompanying Examples, an Fab fragment designated “MOR02683” binds to GFRalpha3 and inhibits formation of a Neublastin-GFRalpha3-Ret ternary complex. The complete amino acid sequence of MOR02683 (as well as the amino acid sequence of the VH region, VL region, CDRs, and framework regions) is provided in Table 1 of Example 1.

Variants of MOR02683 can be prepared that (i) retain the ability to inhibit formation of a Neublastin-GFRalpha3-Ret ternary complex, and (ii) contain one or more amino acid additions, substitutions (e.g., conservative amino acid substitutions), and/or deletions, as compared to the MOR02683 sequence disclosed herein, in a variable region (e.g., a VH region and/or a VL region) and/or in a constant region. For example, a variant of MOR02683 can contain one or more amino acid additions, substitutions, and/or deletions, as compared to the MOR02683 sequence disclosed herein, in one or more CDRs and/or one or more framework regions.

Variants of MOR02683 can be prepared using any of a variety of recombinant DNA techniques. One such technique is site-directed mutagenesis, in which a specific nucleotide (or specific nucleotides) is changed in order to change a single amino acid residue (or multiple amino acid residues) in the MOR02683 sequence. An exemplary commercially available site-directed mutagenesis kit is the “Transformer Site Directed Mutagenesis Kit” sold by Clontech Laboratories (Palo Alto, Calif.).

A conservative substitution is the substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The non-polar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution.

In some embodiments, a variant of MOR02683 contains a VH region that is at least 70%, 80%, 85%, 90%, 95%, 98% or 99% identical to SEQ ID NO:1 and/or a VL region that is at least 70%, 80%, 85%, 90%, 95%, 98% or 99% identical to SEQ ID NO:2.

In some embodiments, a variant of MOR02683 contains a first heavy chain CDR that is at least 70%, 80%, 85%, 90%, 95%, 98% or 99% identical to SEQ ID NO:3, a second heavy chain CDR that is at least 70%, 80%, 85%, 90%, 95%, 98% or 99% identical to SEQ ID NO:4, and/or a third heavy chain CDR that is at least 70%, 80%, 85%, 90%, 95%, 98% or 99% identical to SEQ ID NO:5.

In some embodiments, a variant of MOR02683 contains a first light chain CDR that is at least 70%, 80%, 85%, 90%, 95%, 98% or 99% identical to SEQ ID NO:6, a second light chain CDR that is at least 70%, 80%, 85%, 90%, 95%, 98% or 99% identical to SEQ ID NO:7, and/or a third light chain CDR that is at least 70%, 80%, 85%, 90%, 95%, 98% or 99% identical to SEQ ID NO:8.

Percent identity between amino acid sequences is determined using the BLAST 2.0 program. Sequence comparison is performed using an ungapped alignment and using the default parameters (Blossom 62 matrix, gap existence cost of 11, per residue gap cost of 1, and a lambda ratio of 0.85). The mathematical algorithm used in BLAST programs is described in Altschul et al., 1997, Nucleic Acids Research 25:3389-3402.

### Biological Activities of Anti-GFRalpha3 Antibodies and Antibody Fragments

A biologically active anti-GFRalpha3 antibody or antigen-binding fragment thereof binds to GFRalpha3 and inhibits formation of a Neublastin-GFRalpha3-Ret ternary complex. In some embodiments, an anti-GFRalpha3 antibody or antigen-binding fragment thereof is first generated against a GFRalpha3 sequence and subsequently screened for its ability to inhibit ternary complex formation. In other embodiments, an anti-GFRalpha3 antibody or antigen-binding fragment thereof is generated by synthesizing a variant of MOR02683 and assessing the ability of the variant to, like MOR02683, inhibit formation of the Neublastin-GFRalpha3-Ret ternary complex.

In a ternary complex assay, a Neublastin protein forms a complex with the extracellular domain of Ret and the extracellular domain of GFRalpha3. Soluble forms of Ret and GFRalpha3 can be generated as fusion proteins (e.g., a first fusion protein between the extracellular domain of Ret and placental alkaline phosphatase (Ret-AP) and a second fusion protein between the extracellular domain of GFRalpha3 and the Fc domain of human IgG1) and combined with Neublastin. The ability of an anti-GFRalpha3 antibody or antigen-binding fragment thereof to inhibit formation of the ternary complex can be measured. Exemplary ternary complex assays are described in WO 00/01815 and in Example 2.

Mature wild type human Neublastin is 113 amino acids in length and has the following amino acid sequence: AGGPGSRARAAGARGCRLRSQLVPVRALGLG HRSDELVRFRFCSGSCRRARSPHDLSLASLLGAGALRPPPGSRPVSQPCCRPTR YEAVSFMDVNSTWRTVDRLSATACGCLG (SEQ ID NO:12). The sequence of mature wild type rat Neublastin is described in the accompanying Examples.

The phrase “inhibits formation of a Neublastin-GFRalpha3-Ret ternary complex” refers to a reduction in complex formation as compared to that which occurs in the absence of the anti-GFRalpha3 antibody or antigen-binding fragment thereof. Inhibition does not necessarily indicate a total elimination of complex formation. Inhibition may be a reduction of at least 10%, 20%, 30%, 40%, 50%, 60%, 70%, 80%, 90%, or more. In some embodiments, the anti-GFRalpha3 antibody or antigen-binding fragment thereof inhibits formation of a Neublastin-GFRalpha3-Ret ternary complex, as measured by the ternary complex assay described in Example 2, with an EC₅₀ of 1.0 ug/ml or less (e.g., 0.5 ug/ml or less, 0.25 ug/ml or less, or 0.1 ug/ml or less).

An anti-GFRalpha3 antibody or antigen-binding fragment thereof can also be assessed to evaluate its ability to block triggering of the Neublastin signaling cascade. For example, the Kinase Receptor Activation (KIRA) assay can be used to assess the ability of an anti-GFRalpha3 antibody or antigen-binding fragment thereof to block Neublastin-induced Ret autophosphorylation (see WO 00/01815 and Sadick et al., 1996, Anal. Biochem., 235(2):207). In addition, or alternatively, the phosphorylation status of ERK following administration of Neublastin can be monitored to assess the ability of an anti-GFRalpha3 antibody or antigen-binding fragment thereof to block this pathway. As detailed in the accompanying Examples, an anti-GFRalpha3 antibody or antigen-binding fragment thereof can (in addition to inhibiting Neublastin-GFRalpha3-Ret ternary complex formation) also block Neublastin-induced phosphorylation of Ret and/or block Neublastin-dependent phosphorylation of ERK.

The following is an example of conditions under which a KIRA assay can be performed. Cells expressing Ret and GFRalpha3 are plated at 2×10⁵ cells per well in 24-well plates in Dulbecco's modified eagle medium (DMEM), supplemented with 10% fetal bovine serum, and cultured for 18 hours at 37° C. and 5% CO₂. The cells are then washed with Phosphate Buffered Saline (PBS) and treated with an anti-GFRalpha 3 antibody or antigen-binding fragment thereof and Neublastin in 0.25 mL of DMEM for 10 minutes at 37° C. and 5% CO₂. The cells are washed with 1 mL of PBS, and lysed for 1 hour at 4° C. with 0.30 mL of 10 mM Tris HCl, pH 8.0, 0.5% Nonidet P40, 0.2% sodium deoxycholate, 50 mM NaF, 0.1 mM Na₃ VO₄, 1 mM phenylmethylsulfonyl fluoride with gently rocking the plates. The lysates are further agitated by repeated pipetting and 0.25 mL of sample is transferred to a 96-well ELISA plate that has been coated with 5 ug/mL of anti-Ret monoclonal antibody in 50 mM carbonate buffer, pH 9.6 at 4° C. for 18 h, and blocked at room temperature for one hour with block buffer (20 mM Tris HCl pH 7.5, 150 mM NaCl, 0.1% Tween-20 (TBST) containing 1% normal mouse serum and 3% bovine serum albumin). After a 2 hour incubation at room temperature, the wells are washed 6-times with TBST. Phosphorylated Ret is detected by incubating the wells at room temperature for 2 hours with 2 ug/mL of horseradish peroxidase (HRP)-conjugated anti-phosphotyrosine 4G10 antibody in block buffer, washing 6-times with TBST, and measuring HRP activity at 450 nm with a colorometric detection reagent. The absorbance values from wells treated with lysate or with lysis buffer are measured and the background corrected signal is plotted as a function of the concentration of anti-GFRalpha 3 antibody or antigen-binding fragment thereof present in the mixture.

### Antibody Production

Antibodies can be produced in prokaryotic and eukaryotic cells. In some embodiments, antibodies (e.g., scFv's) are expressed in a yeast cell such as Pichia (see, e.g., Powers et al. (2001) J Immunol Methods. 251:123-35), Hanseula, or Saccharomyces.

In some embodiments, antibodies, particularly full length antibodies, e.g., IgG's, are produced in mammalian cells. Exemplary mammalian host cells for recombinant expression include Chinese Hamster Ovary (CHO cells) (including dhfr- CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NS0 myeloma cells and SP2 cells, COS cells, K562, and a cell from a transgenic animal, e.g., a transgenic mammal. For example, the cell can be a mammary epithelial cell.

In addition to a nucleic acid sequence encoding the immunoglobulin domain, recombinant expression vectors may carry additional nucleic acid sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). Exemplary selectable marker genes include the dihydrofolate reductase (DHFR) gene (for use in dhfr- host cells with methotrexate selection/amplification) and the neo gene (for G418 selection).

In an exemplary system for recombinant expression of an antibody (e.g., a full length antibody or an antigen-binding portion thereof), a recombinant expression vector encoding both the antibody heavy chain and the antibody light chain is introduced into dhfr- CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, to transfect the host cells, to select for transformants, to culture the host cells, and to recover the antibody from the culture medium. For example, some antibodies can be isolated by affinity chromatography with a Protein A or Protein G.

Antibodies may also include modifications, e.g., modifications that alter Fc function, e.g., to decrease or remove interaction with an Fc receptor or with C1q, or both. For example, the human IgG1 constant region can be mutated at one or more residues, e.g., one or more of residues 234 and 237, e.g., according to the numbering in U.S. Pat. No. 5,648,260. Other exemplary modifications include those described in U.S. Pat. No. 5,648,260.

For some antibodies that include an Fc domain, the antibody production system may be designed to synthesize antibodies in which the Fc region is glycosylated. For example, the Fc domain of IgG molecules is glycosylated at asparagine 297 in the CH2 domain. This asparagine is the site for modification with biantennary-type oligosaccharides. This glycosylation participates in effector functions mediated by Fc receptors and complement C1q (Burton and Woof (1992) Adv. Immunol. 51:1-84; Jefferis et al. (1998) Immunol. Rev. 163:59-76). The Fc domain can be produced in a mammalian expression system that appropriately glycosylates the residue corresponding to asparagine 297. The Fc domain can also include other eukaryotic post-translational modifications.

Antibodies can also be produced by a transgenic animal. For example, U.S. Pat. No. 5,849,992 describes a method for expressing an antibody in the mammary gland of a transgenic mammal. A transgene is constructed that includes a milk-specific promoter and nucleic acid sequences encoding the antibody of interest, e.g., an antibody described herein, and a signal sequence for secretion. The milk produced by females of such transgenic mammals includes, secreted-therein, the antibody of interest, e.g., an antibody described herein. The antibody can be purified from the milk, or for some applications, used directly.

Antibodies can be modified, e.g., with a moiety that improves its stabilization and/or retention in circulation, e.g., in blood, serum, lymph, bronchoalveolar lavage, or other tissues, e.g., by at least 1.5, 2, 5, 10, or 50 fold.

In one example, a GFRalpha3 binding antibody can be associated with a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 daltons (or about 1,000 to about 15,000, and 2,000 to about 12,500) can be used.

In another example, a GFRalpha3 binding antibody described herein can be conjugated to a water soluble polymer, e.g., a hydrophilic polyvinyl polymer, e.g. polyvinylalcohol or polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides that comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparon.

### Pharmaceutical Compositions

The anti-GFRalpha3 antibodies and antibody fragments described herein can be administered to a mammalian subject, e.g., a human, alone or in a mixture. For example, the antibodies and antibody fragments can be administered in the presence of a pharmaceutically acceptable excipient or carrier, such as physiological saline. The excipient or carrier is selected on the basis of the mode and route of administration. Suitable pharmaceutical carriers are described in Remington's Pharmaceutical Sciences (E. W. Martin), and in the USP/NF (United States Pharmacopeia and the National Formulary).

A pharmaceutical composition is formulated to be compatible with its intended route of administration. Examples of routes of administration include, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, polypropylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

A pharmaceutical composition may include a “therapeutically effective amount” or a “prophylactically effective amount” of an antibody or antibody fragment described herein. As used herein, “therapeutically effective amount” means an amount effective, at dosages, and for periods of time necessary, to achieve the desired therapeutic result. A therapeutically effective amount of the antibody or antibody fragment can vary according to factors such as the disease state, age, sex, and weight of the individual, and the ability of the antibody, antibody derivative, or antigen-binding polypeptide to elicit a desired response in an individual. When a therapeutically effective amount is administered, any toxic or detrimental effects of the antibody or antibody fragment are outweighed by the therapeutically beneficial effects. As used herein, “prophylactically effective amount” means an amount effective, at dosages, and for periods of time necessary, to achieve the desired prophylactic result.

Dosage regimens can be adjusted to provide the optimum desired response, e.g., a therapeutic or prophylactic response. For example, in some embodiments of the invention a single bolus is administered. In other embodiments, several divided doses are administered over time. The dose can be reduced or increased proportionately, as indicated by the exigencies of the situation. It is advantageous to formulate parenteral compositions in dosage unit form for ease of administration and uniformity of dosage. As used herein, “dosage unit form” means physically discrete units suitable as unitary dosages for the mammalian subjects to be treated, with each containing a predetermined quantity of active ingredient calculated to produce the desired therapeutic effect in association with the required pharmaceutical carrier.

Exemplary, non-limiting ranges for a therapeutically or prophylactically effective amount of an antibody or antibody fragment are 0.1-100 mg/kg, 0.5-50 mg/kg, more 1-20 mg/kg, and 1-10 mg/kg. Dosage values may vary with the type and severity of the condition being treated. For any particular subject, specific dosage regimens can be adjusted over time according to the individual need and the professional judgment of the person administering or supervising the administration of the compositions. It is to be understood that dosage ranges set forth herein are exemplary only and are not intended to limit the scope of the claimed invention.

Parenteral injectable administration can be used for subcutaneous, intramuscular, or intravenous injections and infusions. Additionally, one approach for parenteral administration employs the implantation of a slow-release or sustained-released systems, which assures that a constant level of dosage is maintained, according to U.S. Pat. No. 3,710,795, incorporated herein by reference.

In general, a suitable subject is any mammal to which an anti-GPRalpha3 antibody may be administered. Subjects specifically intended for treatment or prophylaxis include humans, nonhuman primates, sheep, horses, cattle, goats, pigs, dogs, cats, rabbits, guinea pigs, hamsters, gerbils, rats and mice.

### Uses in Binding Assays and Methods of Treatment

Direct binding assays using labeled Neublastin are difficult to perform due to non-specific binding of basic Neublastin to the cell surface. As a solution to this problem, anti-GFRalpha3 antibodies and antigen-binding fragments thereof described herein can be used to detect specific binding of Neublastin to Ret/GFRalpha3 receptors on a cell surface. Because these anti-GFRalpha3 antibodies and antibody fragments only bind to GFRalpha3 receptors that are not in a complex with Neublastin, they can be used to probe for unoccupied GFRalpha3 receptors and thereby measure Neublastin binding affinities to receptors on the cell surface. An exemplary competition binding assay for measuring Neublastin binding affinities is described in Example 5. Anti-GFRalpha3 antibodies described herein can be used to detect binding of a naturally occurring form of Neublastin (e.g., the mature form of human Neublastin described herein) or a biologically active variant of fragment thereof (e.g., a Neublastin variant of fragment as described in WO 00/01815, WO 02/060929, or WO 04/069176).

Ret is a proto-oncogene and has been implicated in the etiology of several human cancers. In addition, its co-receptor GFRalpha3 may be upregulated in some types of cancers (e.g., small cell lung carcinoma). An anti-GFRalpha3 antibody or antigen-binding fragment thereof described herein can thus be used to neutralize Ret signaling through GFRalpha3 and treat cancer in a subject (e.g., a human). Exemplary cancers that can be treated with an anti-GFRalpha3 antibody or antigen-binding fragment thereof described herein include cancers of the gastrointestinal tract (e.g., esophageal or colon cancer) as well as cancers of the bladder, breast, connective tissue, kidney, lung (e.g., small cell lung carcinoma), lymph node, ovary, skin, stomach, testis, and uterus.

An anti-GFRalpha3 antibody or antigen-binding fragment thereof described herein can also be used for modulating metabolism, growth, differentiation, or survival of a nerve or neuronal cell. In particular, anti-GFRalpha3 antibodies can be used to treat or alleviate a neurological disorder in a subject.

The anti-GFRalpha3 antibodies disclosed herein (and pharmaceutical compositions comprising same) can be used in methods for treating a disorder characterized by damage to sensory neurons or retinal ganglion cells, including neurons in the dorsal root ganglia.

In some embodiments, motor neuron diseases such as amyotrophic lateral sclerosis (“ALS”) and spinal muscular atrophy can be treated. In other embodiments, the anti-GFRalpha3 antibodies can be used to enhance nerve recovery following traumatic injury. Alternatively, or in addition, a nerve guidance channel with a matrix containing anti-GFRalpha3 antibodies can be used. Such nerve guidance channels are disclosed, e.g., U.S. Pat. No. 5,834,029.

In some embodiments, the anti-GFRalpha3 antibodies (and pharmaceutical compositions comprising same) are used in the treatment of various disorders in the eye, including photoreceptor loss in the retina in patients afflicted with macular degeneration, retinitis pigmentosa, glaucoma, and similar diseases.

In some embodiments, the anti-GFRalpha3 antibodies (and pharmaceutical compositions comprising same) are used for treating neuropathic pain, for treating tactile allodynia, for reducing loss of pain sensitivity associated with neuropathy, for treating viral infections and viral-associated neuropathies, and for treating painful diabetic neuropathy.

The following are examples of the practice of the invention. They are not to be construed as limiting the scope of the invention in any way.

### EXAMPLES

### Example 1

### Preparation of an Anti-GFRalpha3 Fab Antibody Fragment

The Fab phage display library HuCAL® GOLD (MorphoSys, Inc., Munich, Germany) was screened against the following sequence derived from the extracellular region of murine GFRalpha3: GNSLATENRFVNSCTQARKKCEANPACKAAYQHLGSCTSSLSRPLPLEESAM SADCLEAAEQLRNSSLIDCRCHRRMKHQATCLDIYWTVHPARSLGDYELDVS PYEDTVTSKPWKMNLSKLNMLKPDSDLCLKFAMLCTLHDKCDRLRKAYGE ACSGIRCQRHLCLAQLRSFFEKAAESHAQGLLLCPCAPEDAGCGERRRNTIAP SCALPSVTPNCLDLRSFCRADPLCRSRLMDFQTHCHPMDILGTCATEQSRCLR AYLGLIGTAMTPNFISKVNTTVALSCTCRGSGNLQDECEQLERSFSQNPCLVE AIAAKHRQLFSQDWAD (SEQ ID NO:13). Fab fragments that bound to the GFRalpha3 sequence were characterized. An anti-GFRalpha3 Fab fragment designated MOR02683 was selected for further investigation.

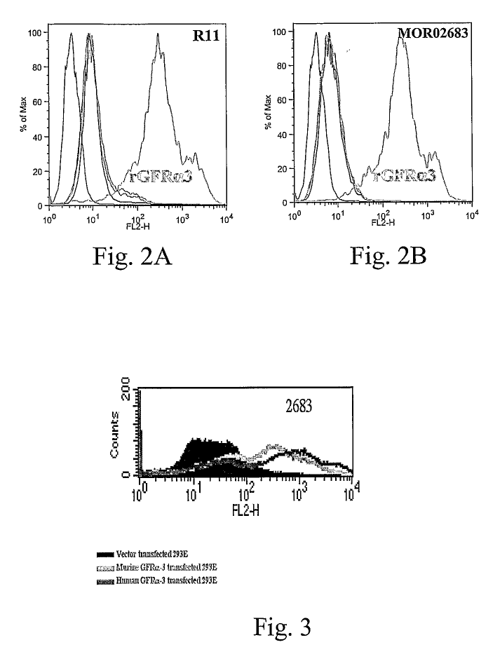

HEK 293 EBNA cells were transiently transfected with an empty vector, a vector encoding rat GFRalpha1, a vector encoding rat GFRalpha2, or a vector encoding rat GFRalpha3. These cells were stained with 10 ug/ml of the anti-GFRalpha3 polyclonal antibody R11 (a rabbit polyclonal antibody generated by immunization with the peptide ARSLGDYELDVSPGC (SEQ ID NO:14), which contains a murine GFRalpha3 sequence and a heterologous GC sequence at its carboxy terminus) or with 10 ug/ml MOR02683. The vector-transfected cells shifted slightly above the unstained baseline because the HEK 293 EBNA cells endogenously expresses GFRalpha3 (FIGS. 2A and 2B). The shifts seen with R11 and MOR02683 in GFRalpha3-transfected cells were comparable, both indicating a strong affinity for the GFRalpha3 receptor (FIGS. 2A and 2B). The lack of shift of the GFRalpha1-transfected cells and GFRalpha2-transfected cells as compared to vector-transfected cells indicated the specificity of MOR02683 for GFRalpha3 over its close family members (FIGS. 2A and 2B).

MOR02683 was also tested on HEK 293 EBNA cells that were transiently transfected with an empty vector, a vector encoding murine GFRalpha□, or a vector encoding human GFRalpha3. FACS analysis indicated that MOR02683 also bound to the human and murine forms of GFRalpha3 (FIG. 3).

In summary, MOR02683 was found to bind to murine, rat, and human GFRalpha3, but not to rat GFRalpha1 or rat GFRalpha2.

The nucleotide and amino acid sequences of the heavy chain and light chain (Kappa 3 family) of the Fab fragment MOR02683 are as follows:

Heavy chain nucleotide sequence

SEQ ID NO:15

(GGAAGCGGCGGCGGCCTGGTGCAACCGGGCGGCAGCCTGCGTCTGAGCT
GCGCGGCCTCCGGATTTACCTTTTCTAATTATACTATGCATTGGGTGCGC
CAAGCCCCTGGGAAGGGTCTCGAGTGGGTGAGCGTTATCTCTTATGATGG
TAGCTCTACCTATTATGCGGATAGCGTGAAAGGCCGTTTTACCATTTCAC
GTGATAATTCGAAAAACACCCTGTATCTGCAAATGAACAGCCTGCGTGCG
GAAGATACGGCCGTGTATTATTGCGCGCGTATTGTTCGTATGGATATTTG
GGGCCAAGGCACCCTGGTGACGGTTAGCTCAGCGTCGACCAAAGGTCCAA
GCGTGTTTCCGCTGGCTCCGAGCAGCAAAAGCACCAGCGGCGGCACGGCT
GCCCTGGGCTGCCTGGTTAAAGATTATTTCCCGGAACCAGTCACCGTGAG
CTGGAACAGCGGGGCGCTGACCAGCGGCGTGCATACCTTTCCGGCGGTGC
TGCAAAGCAGCGGCCTGTATAGCCTGAGCAGCGTTGTGACCGTGCCGAGC
AGCAGCTTAGGCACTCAGACCTATATTTGCAACGTGAACCATAAACCGAG
CAACACCAAAGTGGATAAAAAAGTGGAACCGAAAAGC;);
Heavy chain amino acid sequence

SEQ ID NO:16

(QVQLVESGGGLVQPGGSLRLSCAASGFTFSNYTMHWVRQAPGKGLEWVS
VISYDGSSTYYADSVKGRFTISRDNSKNTLYLQMNSLRAEDTAVYYCARI
VRMDIWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKS;);
Light chain nucleotide sequence

SEQ ID NO:17

(GATATCGTGCTGACCCAGAGCCCGGCGACCCTGAGCCTGTCTCCGGGCG
AACGTGCGACCCTGAGCTGCAGAGCGAGCCAGTCTGTTAATTCTCATTAT
CTGGCTTGGTACCAGCAGAAACCAGGTCAAGCACCGCGTCTATTAATTTA
TGGTGCTTCTAATCGTGCAACTGGGGTCCCGGCGCGTTTTAGCGGCTCTG
GATCCGGCACGGATTTTACCCTGACCATTAGCAGCCTGGAACCTGAAGAC
TTTGCGACTTATTATTGCCAGCAGATGGATGGTTTTCCTTTTACCTTTGG
CCAGGGTACGAAAGTTGAAATTAAACGTACGGTGGCTGCTCCGAGCGTGT
TTATTTTTCCGCCGAGCGATGAACAACTGAAAAGCGGCACGGCGAGCGTG
GTGTGCCTGCTGAACAACTTTTATCCGCGTGAAGCGAAAGTTCAGTGGAA
AGTAGACAACGCGCTGCAAAGCGGCAACAGCCAGGAAAGCGTGACCGAAC
AGGATAGCAAAGATAGCACCTATTCTCTGAGCAGCACCCTGACCCTGAGC
AAAGCGGATTATGAAAAACATAAAGTGTATGCGTGCGAAGTGACCCATCA
AGGTCTGAGCAGCCCGGTGACTAAATCTTTTAATCGTGGCGAGGCC;);
and
Light chain amino acid sequence

SEQ ID NO:18

(DIVLTQSPATLSLSPGERATLSCRASQSVNSHYLAWYQQKPGQAPRLLI
YGASNRATGVPARFSGSGSGTDFTLTISSLEPEDFATYYCQQMDGFPFTF
GQGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTH
QGLSSPVTKSFNRGEA;).

The amino acid sequences of the VH and VL regions, as well as the heavy chain and light chain CDRs, of MOR02683 are detailed in Table 1.

TABLE 1

Amino Acid Sequences of VH, VL, and CDRs of
MOR02683

| | | |
|---|---|---|
| Region | Amino Acid Sequence | SEQ ID NO |

| | | |
|---|---|---|
| VH | QVQLVESGGGLVQPGGSLRLSCAASGFTFSN | SEQ ID NO:1 |
| | YTMHWVRQAPGKGLEWVSVISYDGSSTYYAD | |
| | SVKGRFTISRDNSKNTLYLQMNSLRAEDTAV | |
| | YYCARIVRMDIWGQGTLVTVS | |
| VL | DIVLTQSPATLSLSPGERATLSCRASQSVNS | SEQ ID NO:2 |
| | HYLAWYQQKPGQAPRLLIYGASNRATGVPAL | |
| | RFSGSGSGTDFTLTISSLEPEDFATYYCQQM | |
| | DGFPFTFGQGTKVEIKR | |
| H-CDR1 | GFTFSNYTMH | SEQ ID NO:3 |
| H-CDR2 | VISYDGSSTYYADSVKG | SEQ ID NO:4 |
| H-CDR3 | IVRMDI | SEQ ID NO:5 |
| L-CDR1 | RASQSVNSHYLA | SEQ ID NO:6 |
| L-CDR2 | GASNRAT | SEQ ID NO:7 |
| L-CDR3 | QQMDGFPF | SEQ ID NO:8 |

### Example 2

### MOR02683 Blocks Formation of the Neublastin Signaling Complex

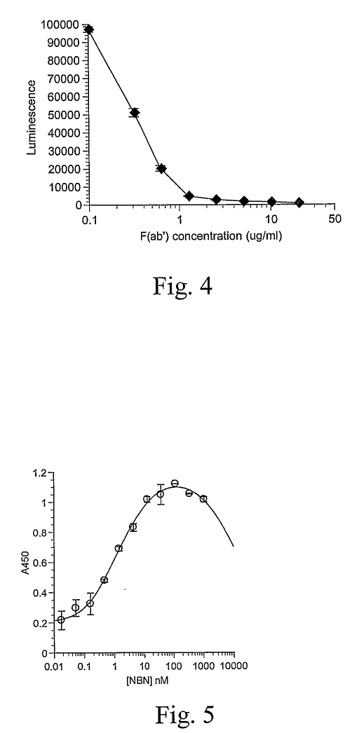

The anti-GFRalpha3 Fab fragment MOR02683 was evaluated for its ability to inhibit formation of a Neublastin-GFRalpha3-Ret ternary complex. Goat anti-human Fc was coated onto a 96-well plate. MOR02683 was preincubated with 1 ug/ml murine GFRalpha3-Ig (MGLSWSPRPPLLMILLLVLSLWLPLGAGNSLATENRFVNSCTQ ARKKCEANPACKAAYQHLGSCTSSLSRPLPLEESAMSADCLEAAEQLRNSSLI DCRCHRRMKHQATCLDIYWTVHPARSLGDYELDVSPYEDTVTSKPWKMNLS KLNMLKPDSDLCLKFAMLCTLHDKCDRLRKAYGEACSGIRCQRHLCLAQLR SFFEKAAESHAQGLLLCPCAPEDAGCGERRRNTIAPSCALPSVTPNCLDLRSFC RADPLCRSRLMDFQTHCHPMDILGTCATEQSRCLRAYLGLIGTAMTPNFISKV NTTVALSCTCRGSGNLQDECEQLERSFSQNPCLVEAIAAKMRQLFSQDW ADVDKTHTCPPCPAPELLGGPSVFLFPPKPKDTLMISRTPEVTCVVVDVSHED PEVKFNWYVDGVEVHNAKTKPREEQYNSTYRVVSVLTVLHQDWLNGKEYK CKVSNKALPAPIEKTISKAKGQPREPQVYTLPPSRDELTKNQVSLTCLVKGFY PSDIAVEWESNGQPENNYKTTPPVLDSDGSFFLYSKLTVDKSRWQQGNVFSC SVMHEALHNHYTQKSLSLSPGK; SEQ ID NO:19) and 50 ng/ml rat Neublastin (113 amino acid form; AGTRSSRARATDARGCRLRSQLVPVSALGLGHSSDELIRFRFCSGSCRRARSP HDLSLASLLDAGALRSPPGSRPISQPCCRPTRYEAVSFMDVNSTWRTVDHLSA TACGCLG; SEQ ID NO:20) in rat Ret-alkaline phosphatase (MAKATSGAAGLGLKLFLLLPLLGEAPLGLYFSRDAYWERLYVDQPAGTPLL YVHALRDAPGEVPSFRLGQYLYGVYRTRLHENDWIHIDAGTGLLYLNQSLDH SSWEQLSIRNGGFPLLTVFLQVFLGSTAQREGECHWPGCARVYFSFINDTFPN CSSFKARDLCTPETGVSFRIRENRPPGTFYQFRMLPVQFLCPNISVKYKLLEGD GLPFRCDPDCLEVSTRWALDRELQEKYVLEAECAVAGPGANKEKVAVSFPV TVYDEDDSPPTFSGGVGTASAVVEFKRKEGTVVATLQVFDADVVPASGELVR RYTSTLLSGDSWAQQTFRVEHTPNETLVQSNNNSVRATMHNYKLVLNRSLSI SESRVLQLVVLVNDSDFQGPGSGVLFLHFNVSVLPVTLNLPMAYSFPVNRRA RRYAQIGKVCVENCQEFSGVSIQYKLQPSSTNCSALGVVTSTEDTSGTLYVND TEALRRPECTELQYTVVATDRQTRRQTQASLVVTVEGTYIAEEVGCPKSCAV NKRRPECEECGGLGSPTGRCEWRQGDGKGITRNFSTCSPSTRTCPDGHCDALE SRDINICPQDCLRGPIVGGHERGERQGIKAGYGICNCFPDEKKCFCEPEDSQGP LCDALCRTVDGGGGIIPVEEENPDFWNREAAEALGAAKKLQPAQTAAKNLIIF LGDGMGVSTVTAARILKGQKKDKLGPEIPLAMDRFPYVALSKTYNVDKHVP DSGATATAYLCGVKGNFQTIGLSAAARFNQCNTTRGNEVISVMNRAKKAGK SVGVVTTTRVQHASPAGTYAHTVNRNWYSDADVPASARQEGCQDIATQLIS NMDIDVILGGGRKYMFPMGTPDPEYPDDYSQGGTRLDGKNLVQEWLAKRQ GARYVWNRTELMQASLDPSVTHLMGLFEPGDMKYEIHRDSTLDPSLMEMTE AALRLLSRNPRGFFLFVEGGRIDHGHHESRAYRALTETIMFDDAIERAGQLTS EEDTLSLVTADHSHVFSFGGYPLRGSSIFGLAPGKARDRKAYTVLLYGNGPG YVLKDGARPDVTESESGSPEYRQQSAVPLDEETHAGEDVAVFARGPQAHLV HGVQEQTFIAHVMAFAACLEPYTACDLAPPAGTTDAAHPG; SEQ ID NO:21) conditioned media for an hour before being added to the coated plate for another hour. The alkaline phosphatase (AP) was visualized with a chemiluminescent substrate and the plate was read on a luminometer. MOR02683 was found to inhibit Neublastin-GFRalpha3-Ret ternary complex formation at an EC₅₀ of about 0.25 ug/ml (FIG. 4).

### Example 3

### MOR02683 Blocks Neublastin-Induced Phosphorylation of Ret

The cell-based Kinase Receptor Activation (KIRA) assay was used to evaluate MOR02683 for its ability to block Neublastin downstream signaling, as measured by Ret phosphorylation. When Neublastin binds to GFRalpha3 (in the absence of a blocking antibody), GFRalpha3 recruits Ret and Ret becomes phosphorylated. The readout for the KIRA assay is Neublastin-induced phosphorylation of Ret.

NB41A3 cells (a murine neuroblastoma cell line; ATCC CCL 147) endogenously expressing murine Ret were stably transfected with a vector encoding murine GFRalpha3 (to generate a cell line designated NB41A3-L3). The cells were preincubated with MOR02683 so that the Fab fragment had the opportunity to bind to GFRalpha3. Three ug/ml of Neublastin was added to the cells for 10 minutes, the cells were lysed, and the lysate was added to a new plate that had been coated with anti-rat Ret antibody (hamster anti-rat Ret monoclonal AA.GE7.3; WO 97/44356). This process traps Ret from the lysate onto the plate. Subsequently, an HRP-tagged anti-phosphotyrosine antibody (recombinant 4G10-HRP conjugate; Catalog Number 16-184; Upstate, Charlottesville, Va.) that binds to plate-bound phosphorylated Ret was added. Binding of the HRP-tagged anti-phosphotyrosine antibody was visualized with an HRP substrate and data was collected from the absorbance of the plate.

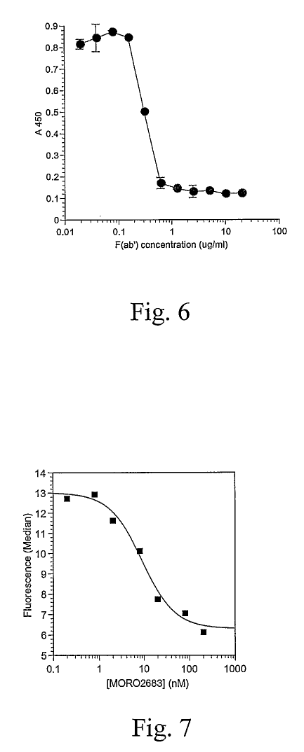

A standard curve of Ret phosphorylation induced by exposure to increasing concentrations of Neublastin confirmed that the assay worked properly (FIG. 5). Addition of MOR02683 was found to inhibit Neublastin-induced Ret phosphorylation in a dose-dependent manner and exhibited and EC₅₀ of about 0.25 ug/ml (FIG. 6).

### Example 4

### MOR02683 Blocks Neublastin-Dependent Phosphorylation of ERK

MOR02683 was evaluated for its ability to block Neublastin downstream signaling, as measured by phosphorylation of the downstream signaling molecule ERK. The phosphorylation of ERK is an event triggered by the activation of the Neublastin/GFRalpha3/Ret complex. NB41A3-L3 cells (expressing Ret and GFRalpha3) were stimulated with 1 nM Neublastin for 10 minutes at 37° C. in the presence of 0-200 nM MOR02683. The amount of phospho-ERK was determined. ERK phosphorylation was inhibited by MOR02683 (IC₅₀=9.1 nM), indicating that MOR02683 blocks Neublastin-dependent phosphorylation of ERK (FIG. 7).

### Example 5

### Neublastin Competition Binding Assay

NB41A3-L3 cells were incubated with different concentrations of rat Neublastin for 10 minutes at 4° C. During the incubation period, the binding reaction of Neublastin to GFRalpha3 and Ret on the cell surface reaches equilibrium. Cells were then quenched with a high concentration (10 ug/ml) of biotinylated MOR02683 for 2 minutes. MOR02683 can only bind to GFRalpha3 receptors that are not already occupied by Neublastin and thus acts as a probe for unoccupied GFRalpha3 receptors. During the short incubation time of the quenching reaction, no re-equilibration between Neublastin and MOR02683 binding occurs. The amount of MOR02683 bound to GFRalpha3 was then quantified via FACS analysis using PE-Streptavidin as a secondary reagent.

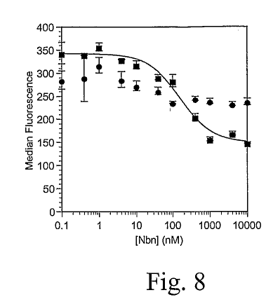

Neublastin competition binding was used to measure Neublastin binding affinities to receptors on the cell surface. NB41A3-L3 cells were incubated with 0-10 uM rat Neublastin for 10 minutes at 4° C. Cells were then quenched with 10 ug/ml biotinylated MOR02683 for 2 minutes. The amount of MOR02683 bound to GFRalpha3 was quantified by FACS using PE-Streptavidin as a secondary reagent. MOR02682, an anti-GFRalpha3 Fab that does not interfere with Neublastin binding, was used as a control. A Neublastin concentration-dependent reduction in the subsequent binding of MOR02683 was observed, but not with the control antibody MOR02682 (FIG. 8). Fitting the data to a hyperbolic equation yielded a Kd of about 200 nM for Neublastin binding to the receptor.

The nucleotide and amino acid sequences of the heavy chain and light chain (Lambda 2 family) of the non-blocking Fab fragment MOR02682 are as follows:

Heavy chain nucleotide sequence

SEQ ID NO:22

(CCCAGGTGCAATTGGTGGAAAGCGGCGGCGGCCTGGTGCAACCGGGCGG
CAGCCTGCGTCTGAGCTGCGCGGCCTCCGGATTTACCTTTAATTCTTATT
GGCTTCATTGGGTGCGCCAAGCCCCTGGGAAGGGTCTCGAGTGGGTGAGC
TCTATCTCTTATTCTGGTAGCAATACCTATTATGCGGATAGCGTGAAAGG
CCGTTTTACCATTTCACGTGATAATTCGAAAAACACCCTGTATCTGCAAA
TGAACAGCCTGCGTGCGGAAGATACGGCCGTGTATTATTGCGCGCGTCAG
CCTACTGCTTCTTTTGATTATTGGGGCCAAGGCACCCTGGTGACGGTTAG
CTCAGCGTCGACCAAAGGTCCAAGCGTGTTTCCGCTGGCTCCGAGCAGCA
AAAGCACCAGCGGCGGCACGGCTGCCCTGGGCTGCCTGGTTAAAGATTAT
TTCCCGGAACCAGTCACCGTGAGCTGGAACAGCGGGGCGCTGACCAGCGG
CGTGCATACCTTTCCGGCGGTGCTGCAAAGCAGCGGCCTGTATAGCCTGA
GCAGCGTTGTGACCGTGCCGAGCAGCAGCTTAGGCACTCAGACCTATATT
TGCAACGTGAACCATAAACCGAGCAACACCAAAGTGGATAAAAAAGTGGA
ACCGAAAAGC;);
Heavy chain amino acid sequence

SEQ ID NO:23

(QVQLVESGGGLVQPGGSLRLSCAASGFTFNSYWLHWVRQAPGKGLEWVS
SISYSGSNTYYADSVKGRFTISRDNSKNTLYLQMNSLRAEDTAVYYCARQ
PTASFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKS;);
Light chain nucleotide sequence

SEQ ID NO:24

(GATATCGCACTGACCCAGCCAGCTTCAGTGAGCGGCTCACCAGGTCAGA
GCATTACCATCTCGTGTACGGGTACTAGCAGCGATATTGGTCGTTATAAT
TTTGTGTCTTGGTACCAGCAGCATCCCGGGAAGGCGCCGAAACTTATGAT
TTATTATGGTAATTCTCGTCCCTCAGGCGTGAGCAACCGTTTTAGCGGAT
CCAAAAGCGGCAACACCGCGAGCCTGACCATTAGCGGCCTGCAAGCGGAA
GACGAAGCGGATTATTATTGCCAGTCTTATGATATGAATAAGCGTGGTTT
TGTGTTTGGCGGCGGCACGAAGTTAACCGTTCTTGGCCAGCCGAAAGCCG
CACCGAGTGTGACGCTGTTTCCGCCGAGCAGCGAAGAATTGCAGGCGAAC
AAAGCGACCCTGGTGTGCCTGATTAGCGACTTTTATCCGGGAGCCGTGAC
AGTGGCCTGGAAGGCAGATAGCAGCCCCGTCAAGGCGGGAGTGGAGACCA
CCACACCCTCCAAACAAAGCAACAACAAGTACGCGGCCAGCAGCTATCTG
AGCCTGACGCCTGAGCAGTGGAAGTCCCACAGAAGCTACAGCTGCCAGGT
CACGCATGAGGGGAGCACCGTGGAAAAAACCGTTGCGCCGACTGAGGC
C;);
and
Light chain amino acid sequence

SEQ ID NO:25

(DIALTQPASVSGSPGQSITISCTGTSSDIGRYNFVSWYQQHPGKAPKLM
IYYGNSRPSGVSNRFSGSKSGNTASLTISGLQAEDEADYYCQSYDMNKRG
FVFGGGTKLTVLGQPKAAPSVTLFPPSSEELQANKATLVCLISDFYPGAV
TVAWKADSSPVKAGVETTTPSKQSNNKYAASSYLSLTPEQWKSHRSYSCQ
VTHEGSTVEKTVAPTEA;).

The amino acid sequences of the VH and VL regions, as well as the heavy chain and light chain CDRs, of MOR02682 are detailed in Table 2.

TABLE 2

Amino Acid Sequences of VH, VL, and CDRs of
MOR02682

| | | |
|---|---|---|
| Region | Amino Acid Sequence | SEQ ID NO |

| | | |
|---|---|---|
| VH | QVQLVESGGGLVQPGGSLRLSCAASGFTFNS | SEQ ID NO:26 |
| | YWLHWVRQALPGKGLEWVSSISYSGSNTYYA | |
| | DSVKGRFTISRDNSKNTLYLQMNSLRAEDTA | |
| | VYYCARQPTASFDYWGQGTLVTVS | |
| VL | DIALTQPASVSGSPGQSITISCTGTSSDIGR | SEQ ID NO:27 |
| | YNFVSWYQQHPGKAPKLMIYYGNSRPSGVSN | |
| | RFSGSKSGNTASLTISGLQAEDEADYYCQSY | |
| | DMNKRGFVFGGGTKLTVL | |
| H-CDR1 | GFTFNSYWLH | SEQ ID NO:28 |
| H-CDR2 | SISYSGSNTYYADSVKG | SEQ ID NO:29 |
| H-CDR3 | QPTASFDY | SEQ ID NO:30 |
| L-CDR1 | TGTSSDIGRYNFVS | SEQ ID NO:31 |
| L-CDR2 | YGNSRPS | SEQ ID NO:32 |
| L-CDR3 | QSYDMNKRGF | SEQ ID NO:33 |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated antibody or antigen-binding fragment thereof that selectively binds to GFRalpha3 and inhibits formation of a Neublastin-GFRalpha3-Ret ternary complex.

2. An isolated antibody or antigen-binding fragment thereof that selectively binds to GFRalpha3 and crossblocks binding of the antibody MOR02683.

3. An isolated antibody or antigen-binding fragment thereof that selectively binds to GFRalpha3 on the same epitope as the antibody MOR02683.

4. An isolated antibody or antigen-binding fragment thereof that selectively binds to GFRalpha and comprises a VH domain that is at least 80% identical to the amino acid sequence of SEQ ID NO:1.

5. The antibody or antigen-binding fragment thereof of claim 4, wherein the VH domain is at least 90% identical to the amino acid sequence of SEQ ID NO:1.

6. The antibody or antigen-binding fragment thereof of claim 4, wherein the VH domain is at least 95% identical to the amino acid sequence of SEQ ID NO:1.

7. The antibody or antigen-binding fragment thereof of claim 4, wherein the VH domain is identical to the amino acid sequence of SEQ ID NO:1.

8. An isolated antibody or antigen-binding fragment thereof that selectively binds to GFRalpha3 and comprises a VL domain that is at least 80% identical to the amino acid sequence of SEQ ID NO:2.

9. The antibody or antigen-binding fragment thereof of claim 8, wherein the VL domain is at least 90% identical to the amino acid sequence of SEQ ID NO:2.

10. The antibody or antigen-binding fragment thereof of claim 8, wherein the VL domain is at least 95% identical to the amino acid sequence of SEQ ID NO:2.

11. The antibody or antigen-binding fragment thereof of claim 8, wherein the VL domain is identical to the amino acid sequence of SEQ ID NO:2.

12. An isolated antibody or antigen-binding fragment thereof that selectively binds to GFRalpha3 and comprises (i) a VH domain that is at least 80% identical to the amino acid sequence of SEQ ID NO:1, and (ii) a VL domain that is at least 80% identical to the amino acid sequence of SEQ ID NO:2.

13. The antibody or antigen-binding fragment thereof of claim 12, wherein (i) the VH domain is at least 90% identical to the amino acid sequence of SEQ ID NO:1, and (ii) the VL domain is at least 90% identical to the amino acid sequence of SEQ ID NO:2.

14. The antibody or antigen-binding fragment thereof of claim 12, wherein (i) the VH domain is at least 95% identical to the amino acid sequence of SEQ ID NO:1, and (ii) the VL domain is at least 95% identical to the amino acid sequence of SEQ ID NO:2.

15. The antibody or antigen-binding fragment thereof of claim 12, wherein (i) the VH domain is identical to the amino acid sequence of SEQ ID NO:1, and (ii) the VL domain is identical to the amino acid sequence of SEQ ID NO:2.

16. An isolated antibody or antigen-binding fragment thereof that selectively binds to GFRalpha3 and comprises a VH domain comprising a heavy chain complementarity determining region (CDR) that is at least 90% identical to the amino acid sequence of SEQ ID NO:3, SEQ ID NO:4, or SEQ ID NO:5.

17. The antibody or antigen-binding fragment thereof of claim 16, wherein the VH domain comprises a first heavy chain CDR that is at least 90% identical to the amino acid sequence of SEQ ID NO:3, a second heavy chain CDR that is at least 90% identical to the amino acid sequence of SEQ ID NO:4, and a third heavy chain CDR that is at least 90% identical to the amino acid sequence of SEQ ID NO:5.

18. The antibody or antigen-binding fragment thereof of claim 16, wherein the VH domain comprises the amino acid sequences of SEQ ID NO:3, SEQ ID NO:4, and SEQ ID NO:5.

19. An isolated antibody or antigen-binding fragment thereof that selectively binds to GFRalpha3 and comprises a VL domain comprising a light chain CDR that is at least 90% identical to the amino acid sequence of SEQ ID NO:6, SEQ ID NO:7, or SEQ ID NO:8.

20. The antibody or antigen-binding fragment thereof of claim 19, wherein the VL domain comprises a first light chain CDR that is at least 90% identical to the amino acid sequence of SEQ ID NO:6, a second light chain CDR that is at least 90% identical to the amino acid sequence of SEQ ID NO:7, and a third light chain CDR that is at least 90% identical to the amino acid sequence of SEQ ID NO:8.

21. The antibody or antigen-binding fragment thereof of claim 19, wherein the VL domain comprises the amino acid sequences of SEQ ID NO:6, SEQ ID NO:7, and SEQ ID NO:8.

22. An isolated antibody or antigen-binding fragment thereof that selectively binds to GFRalpha3 and comprises (i) a VH domain comprising a heavy chain CDR that is at least 90% identical to the amino acid sequence of SEQ ID NO:3, SEQ ID NO:4, or SEQ ID NO:5, and (ii) a VL domain comprising a light chain CDR that is at least 90% identical to the amino acid sequence of SEQ ID NO:6, SEQ ID NO:7, or SEQ ID NO:8.

23. The antibody or antigen-binding fragment thereof of claim 22, wherein (i) the VH domain comprises a first heavy chain CDR that is at least 90% identical to the amino acid sequence of SEQ ID NO:3, a second heavy chain CDR that is at least 90% identical to the amino acid sequence of SEQ ID NO:4, and a third heavy chain CDR that is at least 90% identical to the amino acid sequence of SEQ ID NO:5, and (ii) the VL domain comprises a first Light chain CDR that is at least 90% identical to the amino acid sequence of SEQ ID NO:6, a second light chain CDR that is at least 90% identical to the amino acid sequence of SEQ ID NO:7, and a third light chain CDR that is at least 90% identical to the amino acid sequence of SEQ ID NO:8.

24. The antibody or antigen-binding fragment thereof of claim 22, wherein (i) wherein the VH domain comprises the amino acid sequences of SEQ ID NO:3, SEQ ID NO:4, and SEQ ID NO:5, and (ii) the VL domain comprises the amino acid sequences of SEQ ID NO:6, SEQ ID NO:7, and SEQ ID NO:8.

25. The isolated antibody or antigen-binding fragment thereof of claim 1, wherein the antibody is a humanized antibody.

26. The isolated antibody or antigen-binding fragment thereof of claim 1, wherein the antibody is a fully human antibody.

27. The isolated antibody or antigen-binding fragment thereof of claim 1, wherein the antibody is a monoclonal antibody.

28. The isolated antibody or antigen-binding fragment thereof of claim 1, wherein the antibody is a single chain antibody.

29. The isolated antibody or antigen-binding fragment thereof of claim 1, wherein the antibody or antigen-binding fragment thereof is a polyclonal antibody, a chimeric antibody, an F_{ab} fragment, an F_{(ab′)2} fragment, an F_{ab′} fragment, an F_{sc} fragment, or an Fᵥ fragment.

30. An isolated cell that produces the antibody or antigen-binding fragment thereof of claim 1.

31. The cell of claim 30, wherein the cell is a fused cell obtained by fusing a mammalian B cell and myeloma cell.

32. A pharmaceutical composition comprising the antibody or antigen-binding fragment thereof of claim 1 and a pharmaceutically acceptable carrier.

33. A method of inhibiting formation of a Neublastin-GFRalpha3-Ret ternary complex in a cell, the method comprising contacting a cell expressing GFRalpha3 with an amount of the antibody or antigen-binding fragment thereof of claim 1 effective to inhibit formation of a Neublastin-GFRalpha3-Ret ternary complex.

34. A method of inhibiting Ret phosphorylation in a cell, the method comprising contacting a cell expressing GFRalpha3 with an amount of the antibody or antigen-binding fragment thereof of claim 1 effective to inhibit Ret phosphorylation.

35. A method of treating cancer in a subject, the method comprising administering to a subject in need thereof a pharmaceutical composition comprising an effective amount of the antibody or antigen-binding fragment thereof of claim 1.


SEQUENCE LISTING (33 sequences)

SEQ ID NO: 1 - protein, 114 aa, Artificial Sequence
  Synthetically generated peptide
QVQLVESGGGLVQPGGSLRLSCAASGFTFSNYTMHWVRQAPGKGLEWVSVISYDGSSTYYADSVKGRFTISRDNSKNTLYLQMNSLRAEDTAVYYCARIVRMDIWGQGTLVTVS

SEQ ID NO: 2 - protein, 109 aa, Artificial Sequence
  Synthetically generated peptide
DIVLTQSPATLSLSPGERATLSCRASQSVNSHYLAWYQQKPGQAPRLLIYGASNRATGVPARFSGSGSGTDFTLTISSLEPEDFATYYCQQMDGFPFTFGQGTKVEIKR

SEQ ID NO: 3 - protein, 10 aa, Artificial Sequence
  Synthetically generated peptide
GFTFSNYTMH

SEQ ID NO: 4 - protein, 17 aa, Artificial Sequence
  Synthetically generated peptide
VISYDGSSTYYADSVKG

SEQ ID NO: 5 - protein, 6 aa, Artificial Sequence
  Synthetically generated peptide
IVRMDI

SEQ ID NO: 6 - protein, 12 aa, Artificial Sequence
  Synthetically generated peptide
RASQSVNSHYLA

SEQ ID NO: 7 - protein, 7 aa, Artificial Sequence
  Synthetically generated peptide
GASNRAT

SEQ ID NO: 8 - protein, 8 aa, Artificial Sequence
  Synthetically generated peptide
QQMDGFPF

SEQ ID NO: 9 - protein, 400 aa, Homo sapiens
MVRPLNPRPLPPVVLMLLLLLPPSPLPLAAGDPLPTESRLMNSCLQARRKCQADPTCSAAYHHLDSCTSSISTPLPSEEPSVPADCLEAAQQLRNSSLIGCMCHRRMKNQVACLDIYWTVHRARSLGNYELDVSPYEDTVTSKPWKMNLSKLNMLKPDSDLCLKFAMLCTLNDKCDRLRKAYGEACSGPHCQRHVCLRQLLTFFEKAAEPHAQGLLLCPCAPNDRGCGERRRNTIAPNCALPPVAPNCLELRRLCFSDPLCRSRLVDFQTHCHPMDILGTCATEQSRCLRAYLGLIGTAMTPNFVSNVNTSVALSCTCRGSGNLQEECEMLEGFFSHNPCLTEAIAAKMRFHSQLFSQDWPHPTFAVMAHQNENPAVRPQPWVPSLFSCTLPLILLLSLW

SEQ ID NO: 10 - protein, 397 aa, Rattus norvegicus
MGLSRSPRPPPLVILLLVLSLWLPLGTGNSLPTENRLVNSCTQARKKCEANPACKAAYQHLDSCTPSLSSPLPSGESATSAACLEAAQQLRNSSLIDCRCHRRMKHQATCLDIYWTVHPVRSLGDYELDVSPYEDTVTSKPWKMNLSKLSMLKPDSDLCLKFAMLCTLNDKCDRLRKAYGEACSGIRCQRHLCLAQLRSFFEKAAESHAQGLLLCPCAPEDAGCGERRRNTIAPSCALPSVAPNCLDLRSFCRADPLCRSRLMDFQTHCHPMDILGTCATEQSRCLRAYLGLIGTAMTPNFISKVNTTVALGCTCRGSGNLQDECEQLEKSFSQNPCLMEAIAAKMRFHRQLFSQDWADSTFSVMQQQNSSPALRPQLRLPVLSFFILTLILLQTLW

SEQ ID NO: 11 - protein, 397 aa, Mus musculus
MGLSWSPRPPLLMILLLVLSLWLPLGAGNSLATENRFVNSCTQARKKCEANPACKAAYQHLGSCTSSLSRPLPLEESAMSADCLEAAEQLRNSSLIDCRCHRRMKHQATCLDIYWTVHPARSLGDYELDVSPYEDTVTSKPWKMNLSKLNMLKPDSDLCLKFAMLCTLHDKCDRLRKAYGEACSGIRCQRHLCLAQLRSFFEKAAESHAQGLLLCPCAPEDAGCGERRRNTIAPSCALPSVTPNCLDLRSFCRADPLCRSRLMDFQTHCHPMDILGTCATEQSRCLRAYLGLIGTAMTPNFISKVNTTVALSCTCRGSGNLQDECEQLERSFSQNPCLVEAIAAKMRFHRQLFSQDWADSTFSVVQQQNSNPALRLQPRLPILSFSILPLILLQTLW

SEQ ID NO: 12 - protein, 113 aa, Homo sapiens
AGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 13 - protein, 332 aa, Mus musculus
GNSLATENRFVNSCTQARKKCEANPACKAAYQHLGSCTSSLSRPLPLEESAMSADCLEAAEQLRNSSLIDCRCHRRMKHQATCLDIYWTVHPARSLGDYELDVSPYEDTVTSKPWKMNLSKLNMLKPDSDLCLKFAMLCTLHDKCDRLRKAYGEACSGIRCQRHLCLAQLRSFFEKAAESHAQGLLLCPCAPEDAGCGERRRNTIAPSCALPSVTPNCLDLRSFCRADPLCRSRLMDFQTHCHPMDILGTCATEQSRCLRAYLGLIGTAMTPNFISKVNTTVALSCTCRGSGNLQDECEQLERSFSQNPCLVEAIAAKMRFHRQLFSQDWAD

SEQ ID NO: 14 - protein, 15 aa, Artificial Sequence
  Synthetically generated peptide
ARSLGDYELDVSPGC

SEQ ID NO: 15 - DNA, 636 bp, Artificial Sequence
  Synthetically generated oligonucleotide
ggaagcggcggcggcctggtgcaaccgggcggcagcctgcgtctgagctgcgcggcctccggatttaccttttctaattatactatgcattgggtgcgccaagcccctgggaagggtctcgagtgggtgagcgttatctcttatgatggtagctctacctattatgcggatagcgtgaaaggccgttttaccatttcacgtgataattcgaaaaacaccctgtatctgcaaatgaacagcctgcgtgcggaagatacggccgtgtattattgcgcgcgtattgttcgtatggatatttggggccaaggcaccctggtgacggttagctcagcgtcgaccaaaggtccaagcgtgtttccgctggctccgagcagcaaaagcaccagcggcggcacggctgccctgggctgcctggttaaagattatttcccggaaccagtcaccgtgagctggaacagcggggcgctgaccagcggcgtgcatacctttccggcggtgctgcaaagcagcggcctgtatagcctgagcagcgttgtgaccgtgccgagcagcagcttaggcactcagacctatatttgcaacgtgaaccataaaccgagcaacaccaaagtggataaaaaagtggaaccgaaaagc

SEQ ID NO: 16 - protein, 217 aa, Artificial Sequence
  Synthetically generated peptide
QVQLVESGGGLVQPGGSLRLSCAASGFTFSNYTMHWVRQAPGKGLEWVSVISYDGSSTYYADSVKGRFTISRDNSKNTLYLQMNSLRAEDTAVYYCARIVRMDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 17 - DNA, 645 bp, Artificial Sequence
  Synthetically generated oligonucleotide
gatatcgtgctgacccagagcccggcgaccctgagcctgtctccgggcgaacgtgcgaccctgagctgcagagcgagccagtctgttaattctcattatctggcttggtaccagcagaaaccaggtcaagcaccgcgtctattaatttatggtgcttctaatcgtgcaactggggtcccggcgcgttttagcggctctggatccggcacggattttaccctgaccattagcagcctggaacctgaagactttgcgacttattattgccagcagatggatggttttccttttacctttggccagggtacgaaagttgaaattaaacgtacggtggctgctccgagcgtgtttatttttccgccgagcgatgaacaactgaaaagcggcacggcgagcgtggtgtgcctgctgaacaacttttatccgcgtgaagcgaaagttcagtggaaagtagacaacgcgctgcaaagcggcaacagccaggaaagcgtgaccgaacaggatagcaaagatagcacctattctctgagcagcaccctgaccctgagcaaagcggattatgaaaaacataaagtgtatgcgtgcgaagtgacccatcaaggtctgagcagcccggtgactaaatcttttaatcgtggcgaggcc

SEQ ID NO: 18 - protein, 215 aa, Artificial Sequence
  Synthetically generated peptide
DIVLTQSPATLSLSPGERATLSCRASQSVNSHYLAWYQQKPGQAPRLLIYGASNRATGVPARFSGSGSGTDFTLTISSLEPEDFATYYCQQMDGFPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 19 - protein, 587 aa, Mus musculus
MGLSWSPRPPLLMILLLVLSLWLPLGAGNSLATENRFVNSCTQARKKCEANPACKAAYQHLGSCTSSLSRPLPLEESAMSADCLEAAEQLRNSSLIDCRCHRRMKHQATCLDIYWTVHPARSLGDYELDVSPYEDTVTSKPWKMNLSKLNMLKPDSDLCLKFAMLCTLHDKCDRLRKAYGEACSGIRCQRHLCLAQLRSFFEKAAESHAQGLLLCPCAPEDAGCGERRRNTIAPSCALPSVTPNCLDLRSFCRADPLCRSRLMDFQTHCHPMDILGTCATEQSRCLRAYLGLIGTAMTPNFISKVNTTVALSCTCRGSGNLQDECEQLERSFSQNPCLVEAIAAKMRFHRQLFSQDWADVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 113 aa, Rattus norvegicus
AGTRSSRARATDARGCRLRSQLVPVSALGLGHSSDELIRFRFCSGSCRRARSPHDLSLASLLDAGALRSPPGSRPISQPCCRPTRYEAVSFMDVNSTWRTVDHLSATACGCLG

SEQ ID NO: 21 - protein, 1132 aa, Artificial Sequence
  Synthetically generated oligonucleotide
MAKATSGAAGLGLKLFLLLPLLGEAPLGLYFSRDAYWERLYVDQPAGTPLLYVHALRDAPGEVPSFRLGQYLYGVYRTRLHENDWIHIDAGTGLLYLNQSLDHSSWEQLSIRNGGFPLLTVFLQVFLGSTAQREGECHWPGCARVYFSFINDTFPNCSSFKARDLCTPETGVSFRIRENRPPGTFYQFRMLPVQFLCPNISVKYKLLEGDGLPFRCDPDCLEVSTRWALDRELQEKYVLEAECAVAGPGANKEKVAVSFPVTVYDEDDSPPTFSGGVGTASAVVEFKRKEGTVVATLQVFDADVVPASGELVRRYTSTLLSGDSWAQQTFRVEHTPNETLVQSNNNSVRATMHNYKLVLNRSLSISESRVLQLVVLVNDSDFQGPGSGVLFLHFNVSVLPVTLNLPMAYSFPVNRRARRYAQIGKVCVENCQEFSGVSIQYKLQPSSTNCSALGVVTSTEDTSGTLYVNDTEALRRPECTELQYTVVATDRQTRRQTQASLVVTVEGTYIAEEVGCPKSCAVNKRRPECEECGGLGSPTGRCEWRQGDGKGITRNFSTCSPSTRTCPDGHCDALESRDINICPQDCLRGPIVGGHERGERQGIKAGYGICNCFPDEKKCFCEPEDSQGPLCDALCRTVDGGGGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFPMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPG

SEQ ID NO: 22 - DNA, 659 bp, Artificial Sequence
  Synthetically generated oligonucleotide
cccaggtgcaattggtggaaagcggcggcggcctggtgcaaccgggcggcagcctgcgtctgagctgcgcggcctccggatttacctttaattcttattggcttcattgggtgcgccaagcccctgggaagggtctcgagtgggtgagctctatctcttattctggtagcaatacctattatgcggatagcgtgaaaggccgttttaccatttcacgtgataattcgaaaaacaccctgtatctgcaaatgaacagcctgcgtgcggaagatacggccgtgtattattgcgcgcgtcagcctactgcttcttttgattattggggccaaggcaccctggtgacggttagctcagcgtcgaccaaaggtccaagcgtgtttccgctggctccgagcagcaaaagcaccagcggcggcacggctgccctgggctgcctggttaaagattatttcccggaaccagtcaccgtgagctggaacagcggggcgctgaccagcggcgtgcatacctttccggcggtgctgcaaagcagcggcctgtatagcctgagcagcgttgtgaccgtgccgagcagcagcttaggcactcagacctatatttgcaacgtgaaccataaaccgagcaacaccaaagtggataaaaaagtggaaccgaaaagc

SEQ ID NO: 23 - protein, 219 aa, Artificial Sequence
  Synthetically generated peptide
QVQLVESGGGLVQPGGSLRLSCAASGFTFNSYWLHWVRQAPGKGLEWVSSISYSGSNTYYADSVKGRFTISRDNSKNTLYLQMNSLRAEDTAVYYCARQPTASFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 24 - DNA, 648 bp, Artificial Sequence
  Synthetically generated oligonucleotide
gatatcgcactgacccagccagcttcagtgagcggctcaccaggtcagagcattaccatctcgtgtacgggtactagcagcgatattggtcgttataattttgtgtcttggtaccagcagcatcccgggaaggcgccgaaacttatgatttattatggtaattctcgtccctcaggcgtgagcaaccgttttagcggatccaaaagcggcaacaccgcgagcctgaccattagcggcctgcaagcggaagacgaagcggattattattgccagtcttatgatatgaataagcgtggttttgtgtttggcggcggcacgaagttaaccgttcttggccagccgaaagccgcaccgagtgtgacgctgtttccgccgagcagcgaagaattgcaggcgaacaaagcgaccctggtgtgcctgattagcgacttttatccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaggggagcaccgtggaaaaaaccgttgcgccgactgaggcc

SEQ ID NO: 25 - protein, 216 aa, Artificial Sequence
  Synthetically generated peptide
DIALTQPASVSGSPGQSITISCTGTSSDIGRYNFVSWYQQHPGKAPKLMIYYGNSRPSGVSNRFSGSKSGNTASLTISGLQAEDEADYYCQSYDMNKRGFVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 26 - protein, 116 aa, Artificial Sequence
  Synthetically generated peptide
QVQLVESGGGLVQPGGSLRLSCAASGFTFNSYWLHWVRQAPGKGLEWVSSISYSGSNTYYADSVKGRFTISRDNSKNTLYLQMNSLRAEDTAVYYCARQPTASFDYWGQGTLVTVS

SEQ ID NO: 27 - protein, 111 aa, Artificial Sequence
  Synthetically generated peptide
DIALTQPASVSGSPGQSITISCTGTSSDIGRYNFVSWYQQHPGKAPKLMIYYGNSRPSGVSNRFSGSKSGNTASLTISGLQAEDEADYYCQSYDMNKRGFVFGGGTKLTVL

SEQ ID NO: 28 - protein, 10 aa, Artificial Sequence
  Synthetically generated peptide
GFTFNSYWLH

SEQ ID NO: 29 - protein, 17 aa, Artificial Sequence
  Synthetically generated peptide
SISYSGSNTYYADSVKG

SEQ ID NO: 30 - protein, 8 aa, Artificial Sequence
  Synthetically generated peptide
QPTASFDY

SEQ ID NO: 31 - protein, 14 aa, Artificial Sequence
  Synthetically generated peptide
TGTSSDIGRYNFVS

SEQ ID NO: 32 - protein, 7 aa, Artificial Sequence
  Synthetically generated peptide
YGNSRPS

SEQ ID NO: 33 - protein, 10 aa, Artificial Sequence
  Synthetically generated peptide
QSYDMNKRGF